(19) United States
(12) Patent Application Publication
(10) Pub. No.: US 2017/0058234 A1
(43) Pub. Date: Mar. 2, 2017

(54) COMPOSITION FOR THE ENZYMATIC DEGUMMING OF OIL

(71) Applicant: Clariant Produkte (Deutschland) GmbH, Frankfurt am Main (DE)

(72) Inventors: Ulrich SOHLING, Freising (DE); KIRSTIN SUCK, München (DE); Paul BUBENHEIM, Hamburg (DE)

(21) Appl. No.: 15/119,802

(22) PCT Filed: Feb. 19, 2015
(86) PCT No.: PCT/EP2015/053504

§ 371 Date: Aug. 18, 2016

(30) Foreign Application Priority Data
Feb. 21, 2014 (EP) 14000633.9

(51) Int. Cl. C11B 3/00; C12N 9/16; A23D 9/04; C12N 9/54; C11B 3/04; A23L 5/20; C12N 9/20; C12N 9/52

(52) U.S. Cl. CPC C11B 3/003; C12Y 301/01004; C12Y 301/01032; C12Y 301/04003; C12Y 304/11; C12Y 304/13; C12Y 304/18; C12Y 304/14; C12Y 304/15; C12Y 304/16; C12Y 304/17; C12Y 304/19; C12Y 304/25; C12Y 304/23; C12N 9/20; C12N 9/16; C12N 9/52; C12N 9/54; C11B 3/04; A23L 5/25; A23D 9/04; A23V 2002/00

(57) ABSTRACT

The present invention relates to a composition comprising at least one phospholipid-splitting enzyme and at least one protease. The invention further relates to a method for degumming triglyceride-containing compositions by use of the composition according to the invention and to the use of said composition for degumming triglyceride-containing compositions.

The invention relates to a composition comprising at least one phospholipid-cleaving enzyme and at least one protease. Furthermore, the invention relates to a method for degumming triglyceride-containing compositions using the composition according to the invention, and also to the use of the composition according to the invention for the degumming of triglyceride-containing compositions.

Triglycerides that are obtained from plant raw materials, in particular raw plant oils, contain phosphatides, protein- and carbohydrate-containing substances, plant gum substances as well as colloidal compounds which greatly reduce the shelf life of the oil and reduce the yield of the purified oil. These substances therefore have to be removed.

During the refining of plant oils, the undesired accompanying substances which would render the oil unusable are removed. A distinction is made between chemical and physical refining. Chemical refining consists of the processes 1. degumming, during which phospholipids and metal ions are removed from the oil, 2. neutralization with alkali, in which the fatty acids are extracted, 3. bleaching to remove dyes, further metal ions and remaining gum substances, 4. deodorization, a steam distillation, in which further compounds are removed which adversely affect the odor and the taste of the oil. During physical refining, the deacidification is carried out together with the deodorization at the end of the refining process.

The degumming of the oils can take place by extraction of the phospholipids with water or an aqueous solution of an acid which complexes Ca²⁺ and Mg²⁺ ions, such as e.g. citric acid or phosphoric acid. Often here, firstly an aqueous, so-called predegumming is carried out, with which the water-soluble phospholipids are removed. The term used herein is hydratable phospholipids.

The theme of hydratable and non-hydratable phospholipids is described for example in Nielsen, K., Composition of difficultly extractable soy bean phosphatides, J. Am. Oil. Chem. Soc. 1960, 37, 217-219 and A. J. Dijkstra, Enzymatic degumming, Eur. J. Lipid Sci. Technol. 2010, 112, 1178-1189. These are in particular phosphatidylcholin and phosphatidylinositol. The treatment with dilute aqueous calcium- and magnesium-complexing acids, such as e.g. citric acid or phosphoric acid, leads, according to the prior art, to non-hydratable phospholipids being converted to hydratable phospholipids. A reduction in phosphorus content to 10 or less ppm of phosphorus in the oil must regularly be demonstrated for food applications (according to the prior art determined by ICP-AES analysis of the oil). For oils that are used for example for producing biodiesel, even stricter requirements are imposed. According to the EU standard, the phosphorus content of the biodiesel is limited there to 5 ppm and it is expedient to carry out the phosphorus reduction already on the oil side.

One disadvantage of conventional oil degumming processes is that both the aqueous predegumming and the treatment with aqueous acids lead to oil losses which are caused by the fact that the phospholipids transferred to the water are emulsifiers which emulsify some of the plant oil in the aqueous phase, as a result of which plant oil is lost. These losses can be in the region of a few percent, based on the crude oil originally used. A rule of thumb is that approximately one triglyceride molecule is emulsified for every two molecules of phospholipid (described in WO 08/094847).

The so-called enzymatic degumming avoids several disadvantages of the existing methods and/or improves the extraction methods.

The use of phospholipases, primarily phospholipase A, for the degumming of crude oils is described for example in EP 0513709 B1 (so-called Enzymax® process from Lurgi, Frankfurt). It is assumed that the cleaving of a fatty acid leads to a lysolecithin, which has a significantly lower emulsifying capacity for oil and also has a significantly higher solubility in water. As a result of this, both the oil yield is increased, and the solubility in water of the difficult-to-hydrate phospholipids is improved. The current prior art relating to enzymatic oil degumming is summarized in the two articles by A. J. Dijkstra, Recent developments in edible oil processing, Eur. J. Lipid Sci. Technol. 2009, 111, 857-864, and the article by A. J. Dijkstra, Enzymatic degumming, Eur. J. Lipid Sci. Technol. 2010, 112, 1178-1189. These discuss the advantages and disadvantages of the individual phospholipases for the enzymatic oil degumming and also the pretreatment methods with various acids.

From the point of view of the oil yield, it would be most favorable for the enzymatic degumming to use a highly effective phospholipase C which produces, as product, a diglyceride which is soluble in the oil, and a phosphatidyl radical, such as e.g. phosphatidylcholine (starting from lecithin), which is very readily soluble in water. Such enzymes are described by Verenium in U.S. Pat. No. 7,226,771. In the review article by Dijkstra on the topic of “Enzymatic degumming”, one disadvantage of this system that is listed is that it does not convert all phospholipids, but only phosphatidylcholine and phosphatidylinositol, whereas the difficult-to-hydrate ethanolamines and phosphatidic acids remain untouched. This disadvantage has led to the phospholipase C combining either with phospholipases A or with lipid acyltransferases in subsequent developments. A combination of phospholipases A with phospholipases C for oil degumming is described in WO 08/094847. This patent specification states that the mixing of phospholipase A and phospholipase C on the one hand leads to a synergistic effect for the oil yield, and on the other hand very low phosphorus contents in the oil with tolerable reaction times can be established therewith.

The combination of phospholipase C with lipid acyltransferases is described in WO 2009/081094. Here too, it is stated that the combination of the acyltransferase with the phospholipase C leads to an increase in oil yield. A further variant of enzymatic oil degumming is the enzymatic treatment of the separated-off gum phase after the oil has been degummed by conventional methods such as e.g. with water and/or citric acid. By virtue of this treatment, it is possible to recover some of the plant oil emulsified in the gum phase. This process is discussed for example also in the review article A. J. Dijkstra, Enzymatic degumming, Eur. J. Lipid Sci. Technol. 2010, 112, 1178-1189, p. 1184.

Furthermore, PCT/EP2013/053199 describes a method in which crude oil is degummed with an enzyme combination of a phospholipid-cleaving enzyme and a glycosidase. A further prior art method, which is described in EP 13166529.1, utilizes a phosphatase in the course of an enzymatic degumming.

Finally, the aspect of the sustainability of the use of phospholipases compared to other degumming methods is described in the article L. De Maria & J. Vind & K. M. Oxenbøll & A. Svendsen & S. Patkar, Phospholipases and their industrial applications, Appl Microbiol Biotechnol (2007) 74:290-300, pp. 96 and 97. Using the example of an oil mill which has been converted from a conventional degumming process to a process with phospholipase A and in which 266 000 t of soybean oil are purified per year, it has been shown that 120 000 GJ of energy and 120 000 t of CO₂ equivalents can be saved there per year. The CO₂ equivalents saved there correspond to the emissions from 1600 average inhabitants.

On account of the worldwide increase in the consumption of food oil and the ever increasing use of plant oils as raw materials for the chemical industry and as fuel, there is constantly a further need to improve the degumming of triglyceride-containing compositions, in particular of plant oils and/or plant oil gums.

The inventors of the present application have therefore set themselves the task of developing an alternative enzymatic method to the methods known from the prior art for the degumming of glyceride-containing compositions, in particular crude plant oils, with which the phosphorus content of the triglyceride to be degummed or of the triglyceride-containing composition is further reduced, the oil yield is increased and/or the rate of reaction of the enzymatic degumming is increased. At the same time, this method should permit economical implementation on the industrial scale.

In the context of the present invention, enzyme activity is defined as a chemical reaction that is catalyzed by one or more catalytic proteins (enzymes). In the process, an enzyme substrate is converted to one or more products. Specific enzymes or enzyme compositions have one or even more enzyme activities. A pure enzyme can catalyze e.g. more than one reaction (conversion of a substrate to product(s)), and therefore has more than one enzyme activity. Many enzyme compositions are not biochemically pure products and therefore have a number of enzyme activities. The enzyme activity is connected with the rate of reaction. It indicates how much active enzyme there is in an enzyme composition. The units of enzyme activity are unit (U), where 1 U is defined as the amount of enzyme which converts one micromole of substrate per minute under stated conditions: 1 U=1 μmol/min.

This object has been achieved by a composition comprising a first enzyme component, comprising at least one phospholipid-cleaving enzyme, and a second enzyme component comprising at least one protease (“composition according to the invention”).

In the context of the present invention, the “phospholipid-cleaving enzyme” is preferably a phospholipase which is able to cleave off either a fatty acid radical or a phosphatidyl radical or a head group from a phospholipid. Furthermore, the “phospholipid-cleaving enzyme” is preferably an acyltransferase, in which the cleaving off of the fatty acid radical is combined with a transfer of this radical, followed by an ester formation, with a free sterol in the oil phase.

Phospholipases are enzymes which belong to the group of hydrolases and which hydrolyze the ester bond of phospholipids. Phospholipases are divided according to their regioselectivity in phospholipids into 5 groups:

Phospholipases A1 (PLAT), which cleave the fatty acid in the sn1 position with the formation of the 2-lysophospholipid.

Phospholipases A2 (PLA2), which cleave the fatty acid in the sn2 position with the formation of the 1-lysophospholipid.

Phospholipases C (PLC), which cleave a phosphoric acid monoester.

Phospholipases D (PLD), which cleave or exchange the head group.

Phospholipases B (PLB), which cleave the fatty acid both in the sn1 position and in the sn2 position, with the formation of a 1,2-lysophospholipid.

In the context of the present invention, an acyltransferase is understood as meaning an enzyme which transfers acyl groups, e.g. fatty acids from a phospholipid, to a suitable acceptor, e.g. a sterol, with formation of an ester.

In a preferred embodiment, the present invention relates to a composition in which the first enzyme component is selected from the group consisting of phospholipase A1, phospholipase A2, phospholipase C, phospholipase B, phospholipase D, acyltransferase and mixtures thereof. The enzymes here can originate from any desired organism (e.g. also isolated from a thermophilic organism) or a synthetic source. The enzymes here can be of animal origin, e.g. from the pancreas, of vegetable origin or of microbial origin, e.g. originate from yeast, fungi, algae or bacteria. In the context of the present invention, it is also possible that within the enzyme components in each case enzymes of an identical type but which originate from different sources or species are used. Likewise encompassed are recombinant chimeric fusion proteins of two or more different species with enzymatic activity.

In the context of the present invention, phospholipase A1, phospholipase A2, phospholipase C, phospholipase B, phospholipase D, acyltransferase and mixtures thereof are preferably used from the following species: porcine pancreas, bovine pancreas, snake venom, bee venom, Aspergillus, Bacillus, Citrobacter, Clostridium, Dictyostelium, Edwardsiella, Enterobacter, Escherichia, Erwinia, Fusarium, Klebsiella, Listeria, Mucor, Naja, Neurospora, Pichia, Proteus, Pseudomonas, Providencia, Rhizomucor, Rhizopus, Salmonella, Sclerotinia, Serratia, Shigella, Streptomyces, Thermomyces, Trichoderma, Trichophyton, Whetzelinia, Yersinia.

Particular preference is given to using phospholipase A1, phospholipase A2, phospholipase C, phospholipase B, phospholipase D, acyltransferase and mixtures thereof from Aspergillus awamori, Aspergillus foetidus, Aspergillus fumigatus, Aspergillus japonicus, Aspergillus niger, Aspergillus oryzae, Bacillus alvei, Bacillus amyloliquefaciens, Bacillus anthracis, Bacillus atrophaeus, Bacillus cereus, Bacillus circulans, Bacillus coagulans, Bacillus larvae, Bacillus laterosporus, Bacillus megaterium, Bacillus natto, Bacillus pasteurii, Bacillus pumilus, Bacillus sphaericus, Bacillus sporothermodurans, Bacillus subtilis, Bacillus thuringiensis, Bacillus pseudoanthracis, Citrobacter amalonaticus, Citrobacter braakii, Citrobacter farmeri, Citrobacter freundii, Citrobacter gillenii, Citrobacter koseri, Citrobacter murliniae, Citrobacter rodentium, Citrobacter sedlakii, Citrobacter werkmanii, Citrobacter youngae, Clostridium perfringens, Dictyostelium discoideum, Dictyostelium mucoroides, Dictyostelium polycephalum, Edwardsiella hoshinae, Edwardsiella ictaluri, Edwardsiella tarda, Enterobacter amnigenus, Enterobacter aerogenes, Enterobacter cloacae, Enterobacter gergoviae, Enterobacter intermedius, Enterobacter pyrinus, Escherichia albertii, Escherichia blattae, Escherichia coli, Escherichia fergusonii, Escherichia hermannii, Escherichia senegalensis, Escherichia vulneris, Erwinia amylovora, Erwinia aphidicola, Erwinia billingiae, Erwinia carotovora, Erwinia herbicola, Erwinia oleae, Erwinia mallotivora, Erwinia papayae, Erwinia persicina, Erwinia piriflorinigrans, Erwinia psidii, Erwinia pyrifoliae, Erwinia rhapontici, Erwinia tasmaniensis, Erwinia toletana, Erwinia tracheiphila, Fusarium avenaceum, Fusarium avenaceum, Fusarium chlamydosporum, Fusarium coeruleum, Fusarium culmorum, Fusarium dimerum, Fusarium incarnatum, Fusarium heterosporum, Fusarium moniliforme, Fusarium napiforme, Fusarium oxysporum, Fusarium poae, Fusarium sporotrichiella, Fusarium tricinctum, Fusarium proliferatum, Fusarium sacchari, Fusarium solani, Fusarium sporotrichioides, Fusarium subglutinans, Fusarium tabacinum, Fusarium verticillioides, Klebsiella oxytoca, Klebsiella mobilis, Klebsiella singaporensis, Klebsiella granulomatis, Klebsiella pneumoniae, Klebsiella variicola, Listeria monocytogenes, Mucor amphibiorum, Mucor circinelloides, Mucor hiemalis, Mucor indicus, Mucor javanicus, Mucor mucedo, Mucor paronychius, Mucor piriformis, Mucor subtilissimus, Mucor racemosus, Naja mossambica, Neurospora Africana, Neurospora crassa, Neurospora discrete, Neurospora dodgei, Neurospora galapagosensis, Neurospora intermedia, Neurospora lineolata, Neurospora pannonica, Neurospora sitophila, Neurospora sublineolata, Neurospora terricola, Neurospora tetrasperma, Pichia barkeri, Pichia cactophila, Pichia cecembensis, Pichia cephalocereana, Pichia deserticola, Pichia eremophilia, Pichia exigua, Pichia fermentans, Pichia heedii, Pichia kluyveri, Pichia kudriavzevii, Pichia manshurica, Pichia membranifaciens, Pichia nakasei, Pichia norvegensis, Pichia orientalis, Pichia pastoris (Komagataella pastoris), Pichia pseudocactophila, Pichia scutulata, Pichia sporocuriosa, Pichia terricola, Proteus hauseri, Proteus mirabilis, Proteus myxofaciens, Proteus penneri, Proteus vulgaris, Pseudomonas aeruginosa, Pseudomonas fluorescens, Pseudomonas putida, Pseudomonas syringae, Providencia rettgeri, Providencia stuartii, Rhizomucor endophyticus, Rhizomucor miehei, Rhizomucor pakistanicus, Rhizomucor pusillus, Rhizomucor tauricus, Rhizomucor variabilis, Rhizopus arrhizus, Rhizopus azygosporus, Rhizopus circinans, Rhizopus japonicus, Rhizopus microsporus, Rhizopus nigricans, Rhizopus oligosporus, Rhizopus oryzae, Rhizopus schipperae, Rhizopus sexualis, Rhizopus stolonifer, Rhizopus artocarpi, Salmonella bongori, Salmonella enterica, Salmonella typhimurium, Sclerotinia borealis, Sclerotinia homoeocarpa, Sclerotinia libertiana, Sclerotinia minor, Sclerotinia ricini, Sclerotinia sclerotiorum, Sclerotinia spermophila, Sclerotinia trifoliorum, Serratia entomophila, Serratia ficaria, Serratia fonticola, Serratia grimesii, Serratia liquefaciens, Serratia marcescens, Serratia odorifera, Serratia plymuthica, Serratia proteamaculans, Serratia quinivorans, Serratia rubidaea, Serratia symbiotica, Shigella dysenteriae, Shigella flexneri, Shigella boydii, Shigella sonnei, Streptomyces achromogenes, Streptomyces ambofaciens, Streptomyces aureofaciens, Streptomyces avermitilis, Streptomyces carcinostaticus, Streptomyces cervinus, Streptomyces clavuligerus, Streptomyces coelicolor, Streptomyces coeruleorubidus, Streptomyces davawensis, Streptomyces fradiae, Streptomyces griseus, Streptomyces hygroscopicus, Streptomyces lavendulae, Streptomyces lincolnensis, Streptomyces natalensis, Streptomyces nodosus, Streptomyces noursei, Streptomyces peuceticus, Streptomyces platensis, Streptomyces rimosus, Streptomyces spectabilis, Streptomyces toxytricini, Streptomyces venezuelae, Streptomyces violaceoniger, Streptomyces violaceoruber, Thermomyces lanuginosa, Trichoderma harzianum, Trichoderma koningii, Trichoderma longibrachiatum, Trichoderma pseudokoningii, Trichoderma reesei, Trichoderma viride, Trichophyton concentricum, Trichophyton eboreum, Trichophyton equinum, Trichophyton gourvilii, Trichophyton kanei, Trichophyton megninii, Trichophyton mentagrophytes, Trichophyton phaseoliforme, Trichophyton raubitschekii, Trichophyton rubrum, Trichophyton schoenleinii, Trichophyton simii, Trichophyton soudanense, Trichophyton terrestre, Trichophyton tonsurans, Trichophyton vanbreuseghemii, Trichophyton verrucosum, Trichophyton violaceum, Trichophyton yaoundei, Whetzelinia schlerotiorum, Yersinia aldovae, Yersinia aleksiciae, Yersinia bercovieri, Yersinia enterocolitica, Yersinia frederiksenii, Yersinia intermedia, Yersinia kristensenii, Yersinia massiliensis, Yersinia mollaretii, Yersinia pestis, Yersinia pseudotuberculosis, Yersinia rohdei, Yersinia ruckeri, Yersinia similis.

In a particularly preferred embodiment, phospholipase A₁, phospholipase A₂, phospholipase B, phospholipase C and/or phospholipase D are used which originate from Aspergillus niger, Aspergillus oryzae, Bacillus cereus, Bacillus megaterium, Bacillus subtilis, Citrobacter freundii, Enterobacter aerogenes, Enterobacter cloacae, Edwardsiella tarda, Erwinia herbicola, Escherichia coli, Clostridium perfringens, Dictyostelium discoideum, Fusarium oxysporium, Klebsiella pneumoniae, Listeria monocytogenes, Mucor javanicus, Mucor mucedo, Mucor subtilissimus, Naja mossambica, Neurospora crassa, Pichia pastoris (Komagataella pastoris), Pseudomonas species, Proteus vulgaris, Providencia stuartii, Rhizomucor pusillus, Rhizopus arrhizus, Rhizopus japonicus, Rhizopus stolonifer, Salmonella typhimurium, Serratia marcescens, Serratia liquefaciens, Sclerotinia libertiana, Shigella flexneri, Streptomyces violaceoruber, Trichophyton rubrum, Thermomyces lanuginosus, Trichoderma reesei, Whetzelinia sclerotiorum, Yersinia enterocolitica, porcine pancreas, bovine pancreas, snake venom or bee venom.

The at least one enzyme of the first enzyme component here can originate from identical or different sources, preferably from one or else also from several of the aforementioned organisms, particularly preferably from Aspergillus niger, Aspergillus oryzae, Fusarium oxysporium, Naja mossambica, Pichia pastoris (Komagataella pastoris), Streptomyces violaceoruber, Thermomyces lanuginosus, Trichoderma reesei, porcine pancreas or bovine pancreas.

In the context of the present invention, the term “protease” is understood as meaning one or more enzymes or enzyme compositions from the enzyme class 3.4 (peptide hydrolases). This includes the terms peptidases and/or proteinases. Proteases catalyze the hydrolysis of peptide bonds. The enzymes here can stem from animal origin, e.g. from gastric mucosa, vegetable origin or microbial origin, e.g. from yeast, fungi, algae or bacteria. In particular, it can preferably be one or more enzymes of the following protease enzyme classes: aminopeptidases, aspartate endopeptidases, dipeptidases, dipeptidylpeptidases, tripeptidylpeptidases, peptidyldipeptidases, carboxypeptidase of the serine type, metallocarboxypeptidases, carboxypeptidases of the cysteine type, omegapeptidases, serin endopeptidases, cysteine endopeptidases, asparagine endopeptidases, metalloendopeptidases, threonine endopeptidases, endopeptidases, with particular preference being given to using aspartate endopeptidases, serine endopeptidases or metalloendopeptidases.

In the context of the present invention, preference is given to using the specified proteases from the following species: gastric mucosa from mammals, porcine pancreas, bovine pancreas, Aspergillus, Bacillus, Citrobacter, Clostridium, Dictyostelium, Edwardsiella, Enterobacter, Escherichia, Erwinia, Fusarium, Klebsiella, Listeria, Mucor, Naja, Neurospora, Pichia, Proteus, Pseudomonas, Providencia, Rhizomucor, Rhizopus, Salmonella, Sclerotinia, Serratia, Shigella, Streptomyces, Thermomyces, Trichoderma, Trichophyton, Whetzelinia, Yersinia.

Particular preference is given to using the protease and mixtures thereof from Aspergillus awamori, Aspergillus foetidus, Aspergillus fumigatus, Aspergillus japonicus, Aspergillus niger, Aspergillus oryzae, Aspergillus sojae, Bacillus alvei, Bacillus amyloliquefaciens, Bacillus anthracis, Bacillus atrophaeus, Bacillus cereus, Bacillus circulans, Bacillus coagulans, Bacillus larvae, Bacillus laterosporus, Bacillus megaterium, Bacillus natto, Bacillus pasteurii, Bacillus pumilus, Bacillus sphaericus, Bacillus sporothermodurans, Bacillus subtilis, Bacillus thuringiensis, Bacillus pseudoanthracis, Bacillus polymyxa, Citrobacter amalonaticus, Citrobacter braakii, Citrobacter farmeri, Citrobacter freundii, Citrobacter gillenii, Citrobacter koseri, Citrobacter murliniae, Citrobacter rodentium, Citrobacter sedlakii, Citrobacter werkmanii, Citrobacter youngae, Clostridium perfringens, Dictyostelium discoideum, Dictyostelium mucoroides, Dictyostelium polycephalum, Edwardsiella hoshinae, Edwardsiella ictaluri, Edwardsiella tarda, Enterobacter amnigenus, Enterobacter aerogenes, Enterobacter cloacae, Enterobacter gergoviae, Enterobacter intermedius, Enterobacter pyrinus, Escherichia albertii, Escherichia blattae, Escherichia coli, Escherichia fergusonii, Escherichia hermannii, Escherichia senegalensis, Escherichia vulneris, Erwinia amylovora, Erwinia aphidicola, Erwinia billingiae, Erwinia carotovora, Erwinia herbicola, Erwinia cleae, Erwinia mallotivora, Erwinia papayae, Erwinia persicina, Erwinia piriflorinigrans, Erwinia psidii, Erwinia pyrifoliae, Erwinia rhapontici, Erwinia tasmaniensis, Erwinia toletana, Erwinia tracheiphila, Fusarium avenaceum, Fusarium avenaceum, Fusarium chlamydosporum, Fusarium coeruleum, Fusarium culmorum, Fusarium dimerum, Fusarium incarnatum, Fusarium heterosporum, Fusarium moniliforme, Fusarium napiforme, Fusarium oxysporum, Fusarium poae, Fusarium sporotrichiella, Fusarium tricinctum, Fusarium proliferatum, Fusarium sacchari, Fusarium solani, Fusarium sporotrichioides, Fusarium subglutinans, Fusarium tabacinum, Fusarium verticillioides, Klebsiella oxytoca, Klebsiella mobilis, Klebsiella singaporensis, Klebsiella granulomatis, Klebsiella pneumoniae, Klebsiella variicola, Listeria monocytogenes, Mucor amphibiorum, Mucor circinelloides, Mucor hiemalis, Mucor indicus, Mucor javanicus, Mucor mucedo, Mucor paronychius, Mucor piriformis, Mucor subtilissimus, Mucor racemosus, Naja mossambica, Neurospora Africana, Neurospora crassa, Neurospora discrete, Neurospora dodgei, Neurospora galapagosensis, Neurospora intermedia, Neurospora lineolata, Neurospora pannonica, Neurospora sitophila, Neurospora sublineolata, Neurospora terricola, Neurospora tetrasperma, Pichia barkeri, Pichia cactophila, Pichia cecembensis, Pichia cephalocereana, Pichia deserticola, Pichia eremophilia, Pichia exigua, Pichia fermentans, Pichia heedii, Pichia kluyveri, Pichia kudriavzevii, Pichia manshurica, Pichia membranifaciens, Pichia nakasei, Pichia norvegensis, Pichia orientalis, Pichia pastoris, Pichia pseudocactophila, Pichia scutulata, Pichia sporocuriosa, Pichia terricola, Proteus hauseri, Proteus mirabilis, Proteus myxofaciens, Proteus penneri, Proteus vulgaris, Pseudomonas aeruginosa, Pseudomonas fluorescens, Pseudomonas putida, Pseudomonas syringae, Providencia rettgeri, Providencia stuartii, Rhizomucor endophyticus, Rhizomucor miehei, Rhizomucor pakistanicus, Rhizomucor pusillus, Rhizomucor tauricus, Rhizomucor variabilis, Rhizopus arrhizus, Rhizopus azygosporus, Rhizopus circinans, Rhizopus japonicus, Rhizopus microsporus, Rhizopus nigricans, Rhizopus oligosporus, Rhizopus oryzae, Rhizopus schipperae, Rhizopus sexualis, Rhizopus stonolifer, Rhizopus artocarpi, Salmonella bongori, Salmonella enterica, Salmonella typhimurium, Sclerotinia borealis, Sclerotinia homoeocarpa, Sclerotinia libertiana, Sclerotinia minor, Sclerotinia ricini, Sclerotinia sclerotiorum, Sclerotinia spermophila, Sclerotinia trifoliorum, Serratia entomophila, Serratia ficaria, Serratia fonticola, Serratia grimesii, Serratia liquefaciens, Serratia marcescens, Serratia odorifera, Serratia plymuthica, Serratia proteamaculans, Serratia quinivorans, Serratia rubidaea, Serratia symbiotica, Shigella dysenteriae, Shigella flexneri, Shigella boydii, Shigella sonnei, Streptomyces achromogenes, Streptomyces ambofaciens, Streptomyces aureofaciens, Streptomyces avermitilis, Streptomyces carcinostaticus, Streptomyces cervinus, Streptomyces clavuligerus, Streptomyces coelicolor, Streptomyces coeruleorubidus, Streptomyces davawensis, Streptomyces fradiae, Streptomyces griseus, Streptomyces hygroscopicus, Streptomyces lavendulae, Streptomyces lincolnensis, Streptomyces natalensis, Streptomyces nodosus, Streptomyces noursei, Streptomyces peuceticus, Streptomyces platensis, Streptomyces rimosus, Streptomyces spectabilis, Streptomyces toxytricini, Streptomyces venezuelae, Streptomyces violaceoniger, Streptomyces violaceoruber, Thermomyces lanuginosa, Trichoderma harzianum, Trichoderma koningii, Trichoderma longibrachiatum, Trichoderma pseudokoningii, Trichoderma reesei, Trichoderma viride, Trichophyton concentricum, Trichophyton eboreum, Trichophyton equinum, Trichophyton gourvilii, Trichophyton kanei, Trichophyton megninii, Trichophyton mentagrophytes, Trichophyton phaseoliforme, Trichophyton raubitschekii, Trichophyton rubrum, Trichophyton schoenleinii, Trichophyton simii, Trichophyton soudanense, Trichophyton terrestre, Trichophyton tonsurans, Trichophyton vanbreuseghemii, Trichophyton verrucosum, Trichophyton violaceum, Trichophyton yaoundei, Whetzelinia schlerotiorum, Yersinia aldovae, Yersinia aleksiciae, Yersinia bercovieri, Yersinia enterocolitica, Yersinia frederiksenii, Yersinia intermedia, Yersinia kristensenii, Yersinia massiliensis, Yersinia mollaretii, Yersinia pestis, Yersinia pseudotuberculosis, Yersinia rohdei, Yersinia ruckeri, Yersinia similis.

In the context of the present invention, preference is given to using proteases from the following species: proteases from gastric mucosa of a mammal, porcine pancreas, bovine pancreas, Aspergillus niger, Aspergillus oryzae, Aspergillus saitoi, Aspergillus sojae, Bacillus cereus, Bacillus licheniformis, Bacillus amyloliquefaciens, Bacillus polymyxa, Bacillus subtilis, Escherichia coli, Clostridium perfringens, Pichia pastoris (Komagataella pastoris), Pseudomonas species, Rhizomucor pusillus, Rhizopus arrhizus, Rhizopus japonicus, Rhizopus stolonifer, Salmonella typhimurium, Serratia marcescens, Serratia liquefaciens, Streptomyces griseus, Streptomyces violaceoruber, Thermomyces lanuginosus, Trichoderma reesei, Yersinia enterocolitica.

The at least one protease of the second enzyme component here can originate from identical or different sources, preferably from one or else also from several of the aforementioned organisms.

In a preferred embodiment, the amount of the enzyme(s) of the first enzyme component is selected in the range from 1 ppm to 1000 ppm, more preferably from 1 to 250 ppm, particularly preferably in the range from 5 to 200 ppm, based on the amount of oil. In a further preferred embodiment, the enzyme activity of the second enzyme component is selected in the range from 1 ppm to 1000 ppm, more preferably from 1 to 250 ppm, particularly preferably in the range from 5 to 200 ppm, based on the amount of oil.

In a preferred embodiment, the enzyme activity of the enzyme(s) of the first enzyme component is chosen in the range from 0.01 to 10 units/g of oil, more preferably in the range from 0.1 to 5 units/g of oil, particularly preferably in the range from 0.2 to 3 units/g of oil and most preferably in the range from 0.3 to 2 units/g of oil. In a further preferred embodiment, the enzyme activity of the second enzyme component is chosen in the range from 0.01 to 10 units/g of oil, preferably 0.1 to 5 units/g of oil, and particularly preferably in the range from 0.2 to 3 units/g of oil, and most preferably in the range from 0.3 to 2 units/g of oil. (Unit: international unit for enzyme activity; 1 unit corresponds to the substrate conversion of 1 μmol/min).

In the context of the present invention, particular preference is given to compositions in which the ratio of the enzyme activity of the first enzyme component to the enzyme activity of the second enzyme component is in the range from 0.001:20 to 20:0.001, preferably in the range from 0.1:10 to 10:0.1, further preferably in the range from 0.25:7.5 to 7.5:0.25, further preferably from 0.5:5 to 5:0.5 and most preferably in the range from 1:1 to 1:5.

By observing the ratio, preferred according to the invention, of the first enzyme component to the second enzyme component, it is possible to further reduce the volume of the gum phase. This signifies an increase in oil yield.

The enzymes of the first and/or second enzyme component can be used for example freeze-dried and dissolved in a corresponding enzyme buffer (standard buffers for each enzyme are described in the literature), e.g. citrate buffer 0.1 M, pH 5 or acetate buffer 0.1 M, pH 5. In a preferred embodiment, the enzymes are taken up in enzyme buffer and added to the crude oil. In order to achieve better solubility of the enzymes—in particular in the phospholipase-containing compositions according to the invention—, the addition of organic solvents is also possible. These are used e.g. in the separation of the phospholipids and are described in the literature. Preference is given to using nonpolar organic solvents such as e.g. hexane or acetone or mixtures, preferably in an amount of from 1 to 30% by weight (examples of possible solvents are described in EP 1531182 A2).

In a further preferred embodiment, the first and/or second enzyme component is used in supported form. Support materials preferred in the context of the present invention are inorganic support materials, such as e.g. silica gels, precipitated silicas, silicates or aluminosilicates, and organic support materials, such as e.g. methacrylates or ion exchanger resins. The support materials facilitate the reusability of the enzymes from the oil/water emulsion in a subsequent process step and contribute to the economic feasibility of the process.

In a likewise preferred embodiment, the composition according to the invention comprises one or more further constituents, particularly preferably selected from the group consisting of citrate buffer and acetate buffer.

The inventors of the present composition have surprisingly discovered that a combination of the enzyme components defined above (first and second enzyme component) reduces the emulsifiability of triglycerides in aqueous phases in a particularly effective manner. Consequently, the composition according to the invention can be used particularly advantageously for the degumming of triglyceride-containing compositions such as crude plant oil or else of plant oil gum.

Consequently, in a further aspect, the present invention relates to a method for degumming triglyceride-containing compositions, involving the steps
a) contacting the triglyceride-containing composition with the composition according to the invention as defined above;
b) separating off the gum substances from the triglyceride-containing composition.

Surprisingly, it has been found in this connection that, by virtue of the method according to the invention, it is possible to further reduce the phospholipid content of the triglyceride-containing composition compared to the sole use of phospholipid-cleaving enzyme, to increase the oil yield, to increase the rate of the reaction during the enzymatic degumming, to lower the gum volume and/or to improve the separability of the formed gum phase. Here, the phosphorus value is reduced to below 20 ppm, particularly preferably to below 10 ppm, very particularly preferably to below 4 ppm of phosphorus.

Furthermore, it is possible with the method according to the invention to reduce the calcium and magnesium content of the triglyceride-containing composition, in particular crude plant oil, to below 20 ppm, particularly preferably to below 15 ppm, very particularly preferably to below 10 ppm, likewise preferably to below 8 ppm and most preferably to below 4 ppm. In a further preferred embodiment, the calcium and magnesium content is lowered to below 3 ppm.

The method of the present invention is particularly advantageous in this case since by using the protease the effect of the phospholipid-cleaving enzyme is improved. As a result of using the protease, there is a lowering of the viscosity of the oil gum phase as well as an increase in the mobility of the phospholipids. Also, the accessibility of the phospholipid molecules located at the gum phase/oil interface for the phospholipid-cleaving enzyme is increased.

As a result of the combining according to the invention of at least one phospholipid-cleaving enzyme with at least one protease, it is moreover possible to reduce the metered addition of the phospholipid-cleaving enzymes, such as e.g. phospholipase A1 or A2 optionally combined with phospholipase C, and in so doing to also save costs besides the aforementioned advantages for the process.

In the context of the present invention, the term “triglycerides” is understood as meaning triple esters of glycerol with fatty acids, which are the main constituent of natural fats and oils, be they of vegetable or animal origin. Triglyceride-containing compositions in the context of the present invention include vegetable or animal fats and oils, and mixtures thereof either with one another or else with synthetic or modified fats and oils. The terms are defined in more detail below.

In the context of the present invention, the term “plant oil” is understood as meaning any oil of plant origin. Preferred particularly suitable oils are soybean oil, rapeseed oil, canola oil, sunflower oil, olive oil, palm oil, jatropha oil, false flax oil, cottonseed oil and mixtures thereof. Of particular suitability are “crude plant oils”. The term “crude” refers here to the fact that the oil has still not been subjected to a degumming, neutralization, bleaching and/or deodorizing step. In the context of the method according to the invention, it is also possible that a mixture of two or more crude oils is used or that pretreated, e.g. predegummed and/or preconditioned, oils are treated with the enzymes.

In the context of the present invention, “gum phase”, “gum substances”, “plant oil gum” are understood as meaning all substances which precipitate out as heavy phase from the triglyceride-containing composition following treatment with an acid-containing and/or aqueous solution (Michael Bokisch: Fats and Oils Handbook, AOCS Press, Champaign, Ill., 1998, pages 428-444). The terms “gum phase”, “gum substances”, “plant oil gum” are used synonymously here in the context of the present invention. The use of this plant oil gum as starting material is of importance especially for obtaining lecithin since lecithin is an important constituent of plant oil gum.

In the context of the present invention, the term “predegumming” or “wet degumming” is understood as meaning a treatment of the crude oil with water or an aqueous acid solution in order to remove water-soluble phospholipids as far as possible from the oil. In the context of the present invention, the terms “predegumming” and “wet degumming” are used here synonymously. Also, in the course of a pre- or wet degumming, after adding the acid it is also possible to add alkali in order to neutralize the acid. Before the enzyme is added, the aqueous phase is separated off. After a predegumming, the phosphorus content in the crude oil is reduced from approx. 500-1500 ppm, e.g. for soybean and rapeseed to below 200 ppm in the predegummed oil. As a result of the predegumming, e.g. lecithin can be obtained from the resulting gum phase and/or the gum phase can be processed as feed. The disadvantage of separating off the aqueous phase or reducing the phosphorus content, however, is a loss in yield with regard to the oil. The phosphatides transferring to the aqueous phase have an emulsifying effect and lead to some of the oil being emulsified in the aqueous phase and separated off with it. Subsequently, the oil can be further treated enzymatically.

In the context of the present invention, the term “preconditioning” of the oil is understood as meaning the addition of water and/or an aqueous acid solution to the untreated oil. Then, by adding alkali, e.g. sodium hydroxide solution, a pH is established at which the following enzymatic reaction takes place. Ideally, the optimum pH of 3.5 to 7 for the enzyme reaction is established. However, the aqueous phase is not subsequently separated off, but the enzymes are added directly. The gum substances present thus remain for the time being in the oil or in the emulsion. The aqueous phase and therefore the enzymes are only separated off after the enzymes have acted on the (optionally preconditioned) crude oil.

In a preferred embodiment, water or an aqueous acid solution and optionally alkali can be added to the crude oil in the sense of a preconditioning to neutralize the acid, but the separating-off of the aqueous phase before adding the enzymes is omitted. By dispensing with the separation step prior to adding the enzymes, a further increase in the oil yield is possible. An increase in the oil yield by one percentage point has an enormous economic significance since this percent corresponds to approx. 400 000 t of oil, based on the annual production of e.g. soybean oil. The method according to the invention thus permits, in this preferred embodiment, the direct use of crude oils from soybean or rapeseed with phosphorus contents of 100 to 1500 ppm phosphorus. Moreover, it constitutes a simplification of the method because the separation step before adding the enzyme is omitted.

In a further preferred embodiment, in step a) of the method according to the invention, no additional emulsifiers, such as e.g. sodium docecylsulfate (SDS), are added—apart from any already present emulsifiers, such as e.g. lecithin. Similarly, the method according to the invention preferably dispenses with the addition of salts, such as e.g. calcium chloride (CaCl₂).

For the degumming of soybean oil or rapeseed oil or sunflower oil, particular preference is given to a combination of phospholipase A1 from Thermomyces lanuginosus or Fusarium oxysporium and/or phospholipase A2 from porcine pancreas or bovine pancreas or Trichoderma reesei or Streptomyces violaceoruber or Aspergillus niger and/or phospholipase C from Pichia pastoris with a gastric protease or Bacillus amyloliquefaciens or Bacillus subtilis or Bacillus licheniformis or Aspergillus niger or Aspergillus oryzae.

In the context of the method according to the invention, the “contacting” according to step a) of the method according to the invention can take place in any manner that is known to the person skilled in the art to be suitable for the purpose according to the invention. A preferred type of contacting according to step a) of the method according to the invention here is a mixing of the triglyceride-containing composition and the composition according to the invention.

After the contacting of the triglyceride-containing composition with the composition according to the invention according to step a) of the method according to the invention, the mixture of the triglyceride-containing composition and of the composition according to the invention is preferably stirred, particularly preferably using a paddle stirrer at 200 to 800 rpm, preferably 250 to 600 rpm and most preferably at 300 to 500 rpm.

The temperature of the mixture during the contacting according to step a) of the method according to the invention is preferably in the range from 15 to 99° C., more preferably in the range from 20 to 95° C., further preferably from 22 to 75° C., likewise preferably from 25 to 65° C., further preferably from 30 to 60° C. and most preferably from 32 to 55° C. In this connection, the temperature of the mixture must always be chosen such that the denaturing temperature of the enzymes is not exceeded, preferably the temperature of the mixture is at least 5° C. below the denaturing temperature of the enzymes or.

The contacting time according to step a) of the method according to the invention here is preferably in the range from 1 minute to 12 hours, more preferably from 5 minutes to 10 hours, likewise preferably from 10 minutes to 6 hours, further preferably from 10 minutes to 3 hours.

The pH of the mixture during the contacting according to step a) of the method according to the invention is preferably in the range from pH 3 to pH 7.5, more preferably in the range from pH 4 to pH 6 and particularly preferably in the range from pH 4.0 to pH 5.5.

The contacting according to step a) of the method according to the invention of the triglyceride-containing composition with the first and the second enzyme component of the composition according to the invention can take place here simultaneously, or else successively. If a contacting is carried out successively, it is preferred in the context of the present invention if the triglyceride-containing composition is firstly brought into contact with the second enzyme component. If the triglyceride-containing composition is contacted firstly with the second enzyme component and then with the first enzyme component, it is particularly preferred if, following the addition of the one component, the mixture is stirred for 30 to 300 minutes, preferably 60 to 240 minutes, likewise preferably from 70 to 120 minutes, before the other component is added.

The “separating-off” of the gum substances according to step b) of the method according to the invention can take place in any manner that is known to the person skilled in the art to be suitable for the purpose according to the invention. Preferably, however, the separation takes place via any separators, such as e.g. centrifuges or filtration units. Preferred separators for the method according to the invention are nozzle separators, screw press separators, chamber separators, disk separators, solid-wall disk separators, two-phase decanters, three-phase decanters, three-pillar centrifuges, single-buffer centrifuges, sliding vibratory centrifuges, vibratory centrifuges, solid-wall peeler centrifuges, solid-wall screw centrifugers, tubular centrifuges, basket peeler centrifuges, pusher centrifuges, screen screw centrifuges, swarf centrifuges, inverting filter centrifuges and universal centrifuges. During the centrifugation, a phase separation of the triglyceride-containing composition takes place such that, for example in the preferred embodiment in which crude plant oil is used as triglyceride-containing composition, the treated plant oil, the gum substances and the enzyme composition are present in separate phases which can be easily separated from one another.

In a preferred embodiment, the phase comprising the gum substances and the phase comprising the composition according to the invention is separated off from the treated oil. It is particularly preferred in this connection if the first and/or second enzyme component is separated off at the same time as the gum substances.

Following separation, the enzymes can be regenerated and/or purified and be used for example in a new degumming method. The enzymes can optionally be regenerated via an adsorbent or via a corresponding column chromatographic method. It is a further option to use some of the heavy phase separated off in a further oil degumming of the method according to the invention.

A further preferred embodiment of the present invention, moreover, relates to a method as described above, further involving the step
c) renewed contacting of the triglyceride-containing composition according to step b) with the first and/or second enzyme component.

The “contacting” according to step c) of the method according to the invention preferably takes place here under the same conditions as described above for step a) of the method according to the invention. Here, in a particularly preferred embodiment, the first and/or second enzyme component is subjected to a regeneration prior to the renewed contacting.

In a particularly preferred embodiment, the triglyceride-containing composition is subjected prior to the contacting according to step c) to a preconditioning as defined above.

In a preferred embodiment of the method according to the invention, the contacting according to step c) takes place as already defined above with regard to the contacting according to step a) of the method according to the invention.

In a further aspect, the present invention relates to the use of the composition according to the invention as defined in more detail above for the degumming of triglyceride-containing compositions.

Particularly preferred embodiments of the present invention are described below, although these in no way limit the scope of the present invention but merely serve for further elucidation:

### PREFERRED EMBODIMENT A)

a) contacting the triglyceride-containing composition selected from crude soybean oil, rapeseed oil and/or sunflower oil with a composition comprising a first enzyme component comprising at least one enzyme selected from the group consisting of phospholipase A1, phospholipase A2 and phospholipase C and a second enzyme component comprising at least one enzyme selected from the group of endoproteases;
b) separating off the gum substances from the triglyceride-containing composition by centrifugation.

In a particularly advantageous embodiment of the method according to the invention according to the preferred embodiment A), before step a) of the method, a so-called preconditioning is carried out in which the crude oil is mixed in a separate method step with an amount of from 1.5 to 3 ml/L of oil of organic acid, preferably citric acid. The temperature of the mixture is adjusted here preferably to 35 to 60° C., particularly preferably to 48° C. After a reaction time of 30 minutes to 2 hours, preferably 1 hour, the mixture is adjusted to a pH of 5 by adding a stoichiometric amount of alkali solution, preferably sodium hydroxide solution, in an amount of preferably 0.5 to 2 mol/l, particularly preferably 1 mol/l. Only then is the procedure continued according to step a) of the method according to the invention.

### PREFERRED EMBODIMENT B)

a) contacting the triglyceride-containing composition selected from crude soybean oil and/or rapeseed oil with a composition comprising a first enzyme component comprising at least one enzyme selected from the group consisting of phospholipase A1, phospholipase A2 and phospholipase C and a second enzyme component with at least one enzyme selected from the group of endoproteases, it being particularly preferred if the at least one enzyme of the first enzyme component is present immobilized on a support;
b) separating off the gum substances from the triglyceride-containing composition by centrifugation.

In a particularly advantageous embodiment of the method according to the invention according to the preferred embodiment B), it is particularly preferred that the enzymes of the first and/or second enzyme component are used in an aqueous phase (buffer preferably in the range pH 4.0 to 5.5, particularly preferably pH 4.0-5.0) in a concentration of 0.05 to 5% w/v. The contacting according to step a) takes place here preferably at a temperature of from 22 to 70° C., more preferably 25 to 65° C.

Moreover, the in the preferred embodiment B) of the method according to the invention, a post-degumming is carried out by adding an organic acid and/or alkali solution (after step b)). The temperature of the mixture here is preferably adjusted to 35 to 60° C., particularly preferably 48° C. After a reaction time of 30 minutes to 2 hours, preferably 1 hour, the mixture is adjusted to a pH of 5 by adding an alkali solution, preferably sodium hydroxide solution, in a concentration of preferably 0.5 to 2 mol/l, particularly preferably 1 mol/l.

### PREFERRED EMBODIMENT C)

a) contacting the triglyceride-containing composition selected from plant oil gum with a composition comprising a first enzyme component comprising at least one enzyme selected from the group consisting of phospholipase A1, phospholipase A2 and phospholipase C and a second enzyme component with at least one enzyme selected from the group of proteases, preferably endoproteases, it being particularly preferred if the at least one enzyme of the first enzyme component is present immobilized on a support;
b) separating off the gum substances from the triglyceride-containing composition by centrifugation.

### PREFERRED EMBODIMENT D)

a) contacting the triglyceride-containing composition selected from crude soybean oil and/or crude rapeseed oil with a composition comprising a first enzyme component comprising at least one enzyme selected from the group consisting of phospholipase A1, phospholipase A2 and phospholipase C and a second enzyme component comprising at least one enzyme selected from the group of proteases, preferably endoproteases, it being particularly preferred if the at least one enzyme of the first enzyme component is present immobilized on a support;
b) separating off the gum substances from the triglyceride-containing composition by centrifugation.

In a particularly advantageous embodiment of the method according to the invention according to the preferred embodiment D), it is particularly preferred that a preconditioning is carried out before step a) of the method by mixing the crude oil in a separate method step with an amount of 50-1500 ppm of organic acid, preferably 100-1200 ppm of citric acid. The temperature of the mixture here is adjusted preferably to 40 to 90° C., particularly preferably 45-85° C. After a reaction time of 5 minutes to 2 hours, preferably 10-30 minutes, the mixture is conditioned by adding an alkali solution, preferably sodium hydroxide solution, in an amount of preferably 0.5 to 5 mol/l, particularly preferably 1 mol/l. Only then is the procedure continued according to step a) of the method according to the invention.

Moreover, any phosphatidic acids still dissolved in the triglyceride-containing composition and not cleaved by the phospholipases can be further reduced by reducing the Ca and/or Mg content of the oil treated according to the method of the present invention. Consequently, the above-listed, preferred embodiments A) to D) of the method according to the invention can advantageously also be supplemented by a subsequent step, in which the content of divalent ions and, in parallel to this, the content of P in the oil is further reduced through renewed addition of complexing agents such as e.g. citric acid or phosphoric acid or oxalic acid or lactic acid or malic acid.

### Methods

The following analytical methods were used:

### Determination of the Phosphorus Content in the Plant Oils

Phosphorus was determined by ICP in accordance with DEV E-22.

### Determination of the Calcium and Magnesium Content in the Plant Oils

Calcium and magnesium were determined by ICP in accordance with DEV E-22.

### Determination of the Content of Free Fatty Acids (FFA)

The content of free fatty acids is determined via the consumption of sodium hydroxide or potassium hydroxide via a saponification reaction. The percentage content of free fatty acids in the investigated oil is obtained. The determination was carried out in accordance with DIN 53402 (method DGF C-V 2).

### Determination of the Gum Volume

The gum phase of enzymatically untreated and enzymatically treated gum present in the oil is measured with the help of this determination. A 10 ml glass centrifugal tube is heated to the working temperature of the reaction mixture, and the samples (2×2 ml) are introduced and centrifuged at 3000 rpm at a controlled temperature for at least 4 minutes in order to separate the gum phase from the oil. Samples are taken from the upper oil phases for analysis. For documentation purposes, the result of the phase formation is additionally photographed.

### Variant 1:

The amount of crude oil to be treated, 400 to 600 g, is introduced into a 1000 ml Duran reactor DN120, and samples are taken for analysis. The oil in the Duran reactor is heated by means of a heating plate to a temperature of from 40 to 85° C., in particular 48 to 80° C. After the temperature is reached, the preconditioning is started. For this, a defined amount, dependent on the amount of oil, of citric acid (e.g. 1000 ppm) is metered into the oil. The mixture is then mixed thoroughly for 1 minute by means of an Ultraturrax®. Alternatively, the mixture is incubated for 15 minutes with stirring at about 600 rpm in order to await the reaction of the acid. Then, a defined amount of sodium hydroxide solution (1 mol/L, residual amount to 2% v/v, or 3% v/v minus water from acid addition and enzyme addition) is added until a pH of approx. 4 is reached, and the mixture is incubated with stirring for a further 10 minutes. After cooling to 48° C., the enzyme, the enzyme mixture or the immobilizate, preferably dissolved in water or buffer, is added. The enzyme is stirred in, for which purpose the stirrer speed can be increased temporarily (1 minute at 900 rpm), then stirring is continued at a lower speed.

Samples are taken at defined time intervals. The sample is removed by means of a pipette, transferred to a heated glass centrifugal tube (temperature of the reaction mixture) and centrifuged at 3000 rpm at a controlled temperature for at least 4 minutes in order to separate the gum phase from the oil. For documentation purposes, the result of the phase formation is photographed, and samples of the supernatant are taken to determine the phosphorus, calcium and magnesium content.

### Variant 2:

In a further procedure, phospholipases and additional enzymes in a suitable combination as free enzymes or immobilized enzymes together with an aqueous phase (enzyme buffer, pH 4-5) 0.05 to 5% w/v, are added to the crude oil. The emulsion, consisting of water, enzyme, possibly enzyme supports and oil, is thoroughly mixed. Ideally, the reaction is carried out at a controlled temperature between 20 and 70° C., better between 40 and 65° C. Then, phase separation is awaited, and the solids settle out or can be removed by a standard method known to the person skilled in the art, e.g. by means of centrifugation or filtration. As after-treatment, the oil can be residually degummed with dilute acid (e.g. citric acid) or alkali solution by a method known to the person skilled in the art as “degumming”.

### Variant 3:

In a further procedure, the gum phase is treated with enzymes. Further enzymes besides phospholipases are added to the gum phase obtained by a method known to the person skilled in the art as “degumming”. These can be present in dissolved form in an aqueous phase or suspended in an organic solvent. The mixture is ideally heated to a temperature between 20 and 70° C., better to a temperature between 35 and 60° C. The mixture is thoroughly mixed until the process has finished. This can be monitored by means of viscosity measurements or visually, by dissolution of the otherwise solid gum phase. A phase separation can be achieved by centrifugation; the individual phases can be separated off. As a rule, the upper phase consists of the obtained oil, the middle phase of the phospholipids and the lower phase is an aqueous phase and comprises the enzymes. By reusing the aqueous phase, the enzymes can be recycled and reused. Depending on the content of divalent ions, the oil or the water phase comprising the enzyme can be freed from the ions by adding complexing agents prior to the subsequent use.

### EXAMPLES AND FIGURES

The invention is explained in more detail below by reference to figures and examples. It is emphasized here that the examples and figures are merely illustrative in character and illustrate particularly preferred embodiments of the present invention. Neither examples nor figures limit the scope of the present invention.

### FIGURES

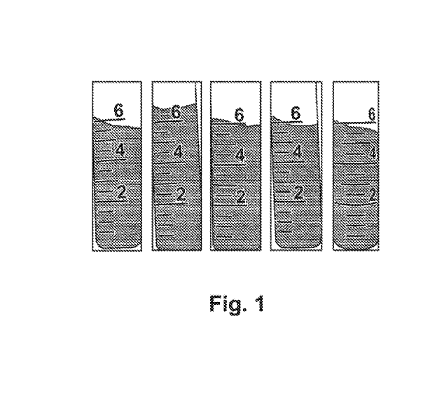

FIG. 1 shows crude rapeseed oil: preconditioning with 3% total water fraction

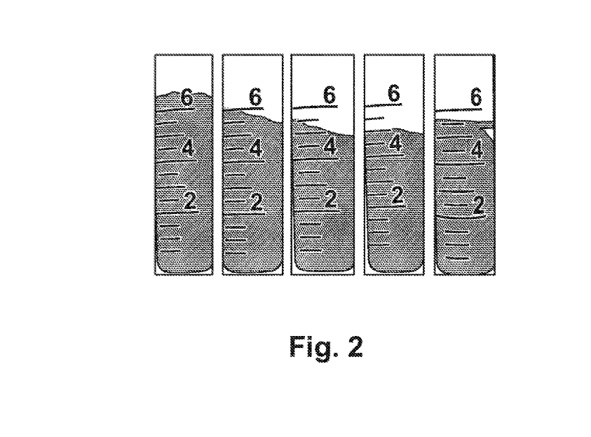

FIG. 2 shows crude rapeseed oil: preconditioning with the addition of enzyme PLA1 0.3 unit/g of oil and 3% total water fraction

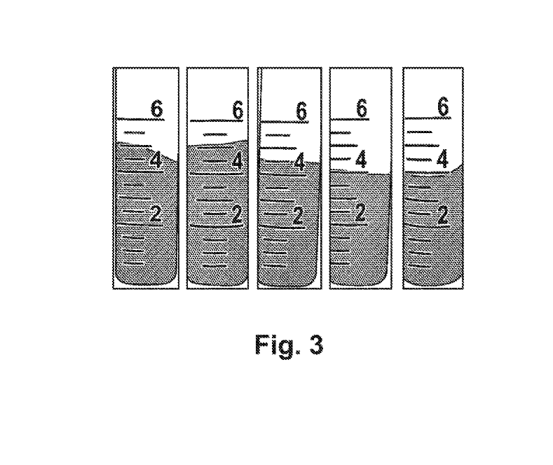

FIG. 3 shows crude rapeseed oil: preconditioning with addition of enzyme PLA1 0.3 unit/g of oil and the enzyme pepsin 1 unit/g of oil, 3% total water fraction

### EXAMPLE 1

According to reaction variant 1, a crude rapeseed oil with the following starting contents was used: phosphorus 1200 ppm, calcium 365 ppm, magnesium 155 ppm and a content of free fatty acids of 1.99%. The crude oil was subjected to a preconditioning with the help of aqueous citric acid (1000 ppm) and aqueous sodium hydroxide solution (1 mol/L). Samples were taken regularly (see FIG. 1, table 1). At the end of the reaction, the gum phase was centrifuged off and the residual oil content of this was determined according to Soxhlet.

As a comparison, just this preconditioning was carried out with the addition of an enzyme, phospholipase A1 from the organism Thermomyces lanuginosus (Sigma-Aldrich) (see FIG. 2, table 2). FIG. 3, table 3 shows the results of the preconditioning with the addition of the enzyme PLAT from Thermomyces lanuginosus and a further enzyme, pepsin from porcine gastric mucosa (Sigma-Aldrich).

TABLE 1
Preconditioning with 3% total water fraction,
phosphorus, calcium, magnesium and FFA content

| Time [min] | 10 | 60 | 120 | 180 | 240 |
|---|---|---|---|---|---|
| Ca [ppm] | 76 | 11 | 9.5 | 9.4 | 9.5 |
| Mg [ppm] | 31 | 2.8 | 1.7 | 1.6 | 1.7 |
| P [ppm] | 247 | 20 | 14 | 13 | 14 |
| FFA [%] | 1.73 | | | 1.68 | 1.72 |
| Gum [%] 3 min | 5.8 | 6.5 | 6.0 | 6.0 | 6.0 |

TABLE 2
Preconditioning with the addition of PLA1 from
Thermomyces lanuginosus 0.3 units/g of oil and 3% total
water fraction, phosphorus, calcium, magnesium and FFA content

| Time [min] | 10 | 60 | 120 | 180 | 240 |
|---|---|---|---|---|---|
| Ca [ppm] | 26 | 9.7 | 8.7 | 7.9 | 7.9 |
| Mg [ppm] | 9.7 | 2.1 | 1.8 | 1.4 | 1.5 |
| P [ppm] | 82 | 17 | 15 | 12 | 12 |
| FFA [%] | 1.76 | | | 2.35 | 2.14 |
| Gum [%] 3 min | 6.5 | 5.6 | 5.0 | 4.5 | 5.5 |

TABLE 3
Preconditioning with the addition of PLA1 (from
Thermomyces lanuginosus) 0.3 units/g of oil and pepsin
1 unit/g of oil, 3% total water fraction, phosphorus,
calcium, magnesium and FFA content

| Time [min] | 10 | 60 | 120 | 180 | 240 |
|---|---|---|---|---|---|
| Ca [ppm] | 55 | 10 | 9.3 | 6.4 | 5.7 |
| Mg [ppm] | 23 | 3.1 | 3 | 1.9 | 1.6 |
| P [ppm] | 199 | 26 | 25 | 16 | 14 |
| FFA [%] | 1.86 | | | 2.14 | 2.17 |
| Gum [%] 3 min | 4.3 | 5.0 | 4.2 | 4.0 | 4.0 |

As is evident from FIG. 1, the use of acid and alkali on the crude oil as preconditioning leads to a not inconsiderable gum volume, which does not subsequently reduce substantially despite using a stirrer at 600 rpm. The only photo corresponds to one sampling. The samplings take place at time points 10, 60, 120, 180 and 240 minutes (from left to right). Table 1 gives the associated analytical data: the phosphorus content dropped after 240 minutes from 247 ppm to 14 ppm; the concentration of the divalent ions calcium and magnesium drops in the case of calcium from 76 ppm to 9.5 ppm; the concentration of the magnesium drops from 31 ppm to 1.7 ppm over the course of the reaction. The content of free fatty acids remains virtually unchanged. The preconditioning serves as preparation reaction for oil degumming and simultaneously as reference treatment.

In FIG. 2, when using the enzyme phospholipase A1 from Thermomyces lanuginosus (Sigma-Aldrich), a decrease in gum volume over the course of the reaction is evident (one photo per measurement/sampling). The associated data and the sampling time points are shown in table 2. Tab. 2 reveals a decrease in the calcium concentration from 26 ppm to 7.9 ppm, a decrease in the magnesium concentration from 9.7 ppm to 1.5 ppm and a decrease in the phosphorus content from 82 ppm to 12 ppm; the content of free fatty acids increases from 1.76% to 2.14%, in each case after a reaction time of 240 min. The increase in the content of the free fatty acids and the decrease in the phosphorus content suggests that the PLA1 is enzymatically active and consequently the oil degumming functions successfully. The increase in the free fatty acid is a sign of the activity of the PLA1, which cleaves the fatty acids from the phospholipid molecules and also the gum volume continuously decreases.

FIG. 3 shows the volume of the gum phase of a preconditioned crude oil treated with PLA1 and additionally with pepsin. It is evident from the associated analytical data in table 3 that surprisingly after just 120 minutes a reduced gum volume of 4.2% is reached, compared to the gum volume of 5.0% when using the PLA1 on its own (table 2). Additionally, the ionic values (table 3) are comparable with those of the reaction with the PLA1 on its own, see table 2. The content of free fatty acids increases from 1.86% to 2.17% and thus points to the activity of the phospholipase. The results show that surprisingly the addition of a single further enzyme, a pepsin, leads to a greater reduction in the gum phase and consequently the oil yield of the reaction is increased.

What is claimed is:

1. A composition comprising:
a first enzyme component comprising at least one phospholipid-cleaving enzyme, and a second enzyme component comprising at least one protease.

2. The composition as claimed in claim 1, wherein the first enzyme component is selected from the group consisting of phospholipase A1, phospholipase A2, phospholipase C, phospholipase B, phospholipase D, acyltransferase and mixtures thereof.

3. The composition as claimed in claim 1, wherein the second enzyme component is selected from the group consisting of aminopeptidases, dipeptidases, dipeptidylpeptidases, tripeptidylpeptidases, peptidyldipeptidases, carboxypeptidase of the serin type, metallocarboxypeptidases, carboxypeptidases of the cystein type, omegapeptidases, serin endopeptidases, cystein endopeptidases, asparagin endopeptidases, metalloendopeptidases, threonin endopeptidases, endopeptidases and mixtures thereof.

4. The composition as claimed in claim 1, wherein the ratio of the first enzyme component to the second enzyme component is in the range from 0.001:20 to 20:0.001.

5. A method for degumming triglyceride-containing compositions, comprising:
a) contacting the triglyceride-containing composition with a composition, said composition comprising a first enzyme component comprising at least one phospholipid-cleaving enzyme, and a second enzyme component comprising at least one protease.
b) separating off the gum substances from the triglyceride-containing composition.

6. The method as claimed in claim 5, wherein the triglyceride-containing composition is selected from crude plant oil and plant oil gum.

7. The method as claimed in claim 5, wherein, before the contacting according to step a), the triglyceride-containing composition is brought into contact with water and/or aqueous acid, but no separation of the aqueous phase takes place before step a).

8. The method as claimed in claim 5, wherein the first and/or second enzyme component is used in supported form.

9. The method as claimed in claim 5, wherein the contacting according to step a) of the method according to the invention takes place at a temperature from 22 to 70° C.

10. The method as claimed in claim 5, further involving the step
c) renewed contacting of the triglyceride-containing composition according to step b) with the first and/or second enzyme component.

11. The use of a composition as defined in claim 1 for the degumming of triglyceride-containing compositions.
